(19) United States
(12) Patent Application Publication
(10) Pub. No.: US 2010/0183752 A1
(43) Pub. Date: Jul. 22, 2010

(54) Foamable Compositions

(76) Inventors: Jean-Paul H. Lecomte, Bruxelles( Auderghem) (BE); Sylvie Bouzeloc, Montigny-Le-Tilleul (BE)

Correspondence Address: DOW CORNING CORPORATION CO1232, 2200 W. SALZBURG ROAD, P.O. BOX 994, MIDLAND, MI, 48686-0994, US

(21) Appl. No.: 12/601,833

(22) PCT Filed: Jun. 27, 2008
(86) PCT No.: PCT/EP2008/058284

§ 371 Date: Nov. 25, 2009

(30) Foreign Application Priority Data
Jul. 2, 2007 (GB) 0712767.3

(51) Int. Cl. A61K 8/97; A61Q 19/00

(52) U.S. Cl. 424/727; 424/725; 424/757; 424/764; 424/731; 424/735; 424/750; 424/771; 424/768

(57) ABSTRACT

A propellant-free foamable transparent composition, foamable by mixing with air or by hand rubbing, comprises a water-immiscible oil composition comprising at least one water-immiscible oil, which may be a mineral oil or vegetable oil, a siloxane resin foam stabiliser which is miscible in the water-immiscible oil composition to form a transparent liquid, and potentially a siloxane resin solubilising agent. The composition may be used for cosmetic applications.

This invention relates to oil-based foamable compositions capable of being foamed by mixing with air by mechanical action, for example by the use of a packaging using a mechanical pump which, upon activation, incorporates air into the liquid material, or by hand rubbing.

Surfactants containing fluoroalkyl lyophobic groups may lower the surface tension of an organic liquid below 21 mN/m, which is lower than any other known solute. Because of their high surface activity, nonionic fluoroalkyl containing surfactants are able to lower the surface tension and stabilize non-aqueous liquid foams. There is however a desire to avoid fluoroalkyl containing compounds for some uses, particularly in personal care products.

U.S. Pat. No. 3,770,648 describes a substantially anhydrous aerosol foaming composition comprising a non-aqueous liquid, a high pressure propellant, classified as fluorocarbon propellant and liquefied petroleum gases and a silicone resin as foaming agent. The propellant is said to be necessary to ensure the production of a proper foam, and solutions of silicone resin in organic solvent carriers are said to be soluble in the propellant. U.S. Pat. No. 4,639,367 describes edible anhydrous aerosol foam composition using lecithin, glycerol ester, polyglycerol ester or sorbitan ester as foaming agent, also in the presence of propellant. The present application relates to compositions which are free from propellant.

WO2005/063198 relates to substantially anhydrous skin cleansing compositions, comprising at least one water insoluble emollient oil and an ionic surfactant in addition to other ingredients. The water insoluble emollient oil is selected from on or more of a number of oils, including mineral oils, vegetable oils and silicone oils, more specifically dimethyl polysiloxane and cyclomethicones. The specification discloses compositions which are able to generate foam upon addition of water, for which purpose the ionic surfactant is added. The present application relates to compositions that are foamable by mixing with air, comprising a siloxane resin which are free from ionic surfactant and do not require addition of water.

JP-A-7-258033 describes a water-in-oil emulsion make-up cosmetic material consisting of a foam progenitor solution containing a liquid oil and a silicone resin, together with water, a surfactant, a colorant and a spraying agent (propellant). JP-A-3-133915 describes a similar composition. JP-A-7-258034 describes a non-emulsion make-up cosmetic material consisting of a foam progenitor solution containing a liquid oil, a silicone resin, a higher alcohol soluble at ambient temperature, a surfactant, a colorant and a spraying agent.

We have found that siloxane resins cause the formation of stable foams of oil-based compositions in the absence of any propellant or spraying agent.

According to the present application a propellant-free foamable transparent composition is provided, which is foamable by mixing with air and comprises a water-immiscible oil composition comprising at least one water-immiscible oil and a siloxane resin foam stabilizer which is miscible with the water-immiscible oil composition to form a transparent liquid.

The water-immiscible oil composition used in a propellant-free foamable transparent composition according to the invention, may comprise a mineral oil, a vegetable oil, or a mixture of one or more of either or both these. The water-immiscible oil composition is preferably free of silicone oils. Siloxane resins are generally insoluble in mineral oils although they do stabilise foam formed from a mineral oil-based composition. Siloxane resins are generally insoluble in vegetable oil and, unless solubilised, do not stabilise foam in vegetable oil. The oil is preferably selected in function of the desired application of the foamable composition; for example it may be an emollient or a moisturiser in a skin conditioning composition.

Where the water-immiscible oil is a mineral oil, this is preferably an aliphatic oil, for example of the type known as liquid paraffin or liquid petrolatum. The mineral oil may alternatively be a hydrocarbon polymer, for example hydrogenated polyisobutylene.

Where the water-immiscible oil is a vegetable oil, this is generally a mixture of fatty acid triglycerides and may for example be coconut oil, groundnut oil, soybean oil, sunflower oil, olive oil, hydrogenated castor oil, sweet almond oil, calophyllum, palm oil, castor oil, avocado oil, jojoba oil, cereal germ oil, corn oil, cottonseed oil, walnut oil, linseed oil, mink oil, illipe butter, rapeseed oil, soybean oil, sunflower seed oil, blackcurrant oil, borage oil, camelina oil, evening primrose oil, rice bran oil or flaxseed oil.

The water-immiscible oil is generally a non-volatile material, for example having a vapour pressure of less than 1 mm Hg at a temperature of 135° C.

The siloxane resin foam stabilizer for use in a propellant-free foamable transparent composition according to the invention, hereafter referred to as siloxane resin, is generally non-linear and consists of siloxane units of the general formula RₐSiO_{4-a/2} wherein R denotes a hydroxyl, hydrocarbon or hydrocarbonoxy group, and wherein a has an average value of from 0.5 to 2.4. It preferably consists predominantly of monovalent trihydrocarbonsiloxy (M) groups of the formula R″₃SiO_{1/2} and tetravalent (Q) groups SiO_{4/2} wherein R″ denotes a monovalent hydrocarbon group, most preferably all siloxane groups are M groups and Q groups as described above. Such most preferable siloxane resins may be called MQ resins. The number ratio of M groups to Q groups is preferably in the range 0.4:1 to 1.2:1 (equivalent to an average value of a in the formula RₐSiO_{4-a/2} of from 0.86 to 1.64). These most preferable MQ resins are solid at room temperature. It is particularly preferred that the number ratio of M groups to Q groups is from 0.5:1 to 0.8:1 (equivalent to an average value of a in the formula RₐSiO_{4-a/2} of from 1.0 to 1.33).

Although it is most preferred that the siloxane resin consists only of monovalent and tetravalent siloxane units as defined above, a siloxane resin comprising M groups, trivalent R″SiO_{3/2} (T) groups and Q groups may alternatively be used. It is also acceptable that in addition up to 20% of all siloxane groups present may be divalent groups R″₂SiO_{2/2} (D). The group R″ is as described above and is preferably in any group of the siloxane resin an alkyl group having 1 to 6 carbon atoms, for example methyl or ethyl, or may be phenyl. It is particularly preferred that at least 80%, most preferably substantially all R″ groups present are methyl groups. Other hydrocarbon groups may be present on some siloxane groups in the siloxane resin, e.g. alkenyl groups present for example as in dimethylvinylsiloxane groups. It is preferred that such other hydrocarbon groups do not exceed 5% of all R″ groups in the siloxane resin. Silicon-bonded hydroxyl groups and/or alkoxy, e.g. methoxy groups may also be present in the siloxane resin, however, it is preferred they do not exceed 5%; preferably not exceed 1% of all silicone bonded substituents in the siloxane resin.

Siloxane resins may be soluble in the water-immiscible oil composition. They may be soluble in the water-immiscible oil or where more than one water-immiscible oil is used, in each water-immiscible oil, for example in a mineral oil or vegetable oil. When using such soluble resins, foamable compositions according to the invention may be easily produced as the mixing of the siloxane resin and the water-immiscible oil composition will be easily accomplished because of the solubility of the siloxane resin in the water-immiscible oil or oils.

Other siloxane resins, in particular the preferred MQ resins, are often insoluble or substantially insoluble in water-immiscible oils such as mineral oil or vegetable oil. We have found that such resins are often more effective in producing a foam of the desired stability, that is a foam which does not immediately collapse on standing in air but may be broken by continued mechanical action. Preferably, the foam does not totally or substantially collapse within 5 seconds, more preferably within 5 minutes, most preferably within 15 minutes on standing in air. The siloxane resin is generally used at 0.5 to 30% by weight of the total foamable composition according to the invention, preferably from 1 or 2 up to 20% by weight.

Such insoluble siloxane resin is therefore preferably used in conjunction with a solubilising agent. The propellant-free foamable transparent composition may accordingly comprise a water-immiscible oil composition comprising at least one mineral and/or vegetable oil, a siloxane resin foam stabiliser and a solubilising agent, whereby the water-immiscible oil composition, the siloxane resin and the solubilising agent are miscible to form a transparent liquid. The siloxane resin may be soluble in the solubilising agent, but this is not essential, provided that the water-immiscible oil composition, the siloxane resin and the solubilising agent are miscible to form a transparent liquid.

One preferred type of solubilising agent is a compound containing at least one ester group. The solubilising agent may for example be an alkyl ester of an aliphatic carboxylic acid having 12 to 20 carbon atoms. Particularly preferred solubilising agents include such esters in which the alkyl group has 4 to 12 carbon atoms. Siloxane resins, including MQ resins, are generally soluble in such esters, for example in octyl stearate, 2-ethylhexyl stearate, decyl isostearate, isodecyl oleate or octyl palmitate. The ester may be an ester of mixed carboxylic acids derived from a vegetable oil, for example a cocoate such as octyl cocoate containing C₁₂ and C₁₄ carboxylates derived from coconut oil. The alkyl group of the ester may alternatively have more than 12 carbon atoms, for example the solubilising ester may be cetyl palmitate, cetyl ricinoleate, cetyl stearate, cetyl myristate, myristyl myristate or cetearyl isononanoate, or less than 4 atoms, for example isopropyl myristate or isopropyl palmitate.

The solubilising agent may alternatively be an alkyl ester of a different carboxylic acid in which the alkyl group has 10 to 20 carbon atoms; the carboxylic acid may for example be an aliphatic carboxylic acid having 2 to 11 carbon atoms or an aromatic or alkaryl carboxylic acid such as a benzoate. The solubilising agent may be a C₁₂₋₁₅ alkyl benzoate, for example those sold under the trademarks Crodamol AB, Finsolv TN or Tegosoft TN, isopropyl palmitate or isopropyl myristate. Further examples of long chain alkyl ester solubilising agents include isotridecyl isononanoate, isostearyl neopentanoate, cetyl octanoate, isodecyl neopentanoate, tridecyl octanoate or cetyl acetate. The alkyl group may contain one or more ether linkages, for example dipropylene glyocol myristyl ether propionate. Other ester solubilising agents include isohexyl neopentanoate, octyl pelargonate, isononyl isononanoate.

An ester solubilising agent may be a monoester as described above or may be a diester, for example propylene dipelargonate, dicaprylyl carbonate, dioctyl malate, polyglyceryl 3-diisostearate, di(tripropylene glycol myristyl ether) adipate, or a polyethylene glycol diheptanoate having 4 oxyethylene units in the polyethylene glycol moiety, or a triester, for example glyceryl trioctanoate or the triglyceride of a mixture of caprylic and caproic acids.

Another preferred type of solubilising agent is a fatty alcohol having for example 12 to 20 carbon atoms, for example octyldodemayol, acetylated lanolin alcohol, isododemayol. Mixtures of two or more of the above ester and/or alcohol stabilisers may be used.

In many propellant-free foamable transparent compositions according to the invention, where solubilising agents are included, it is preferred that the solubilising agent is non-volatile and remains in the composition. However the solubilising agent may, if desired, be volatile, that is it may have a vapour pressure greater than 1 mm Hg at a temperature of 135° C. Such a volatile solubilising agent may be an ester or an alcohol, or may be an aliphatic hydrocarbon or hydrocarbon mixture. For example the C₁₃₋₁₅ alkane sold under the trademark Gemseal 25 is a solvent for many MQ siloxane resins and may be used as a solubilising agent in a composition according to the invention. It is particularly advantageous for use with a water-immiscible oil which is a mineral oil.

The siloxane resin and solubilising agent are preferably present in the composition in a weight ratio in the range from 1:25 to 5:1, preferably 1:10 to 4:1, most preferably 1:1 to 4:1. Some esters effective as solubilising agents are also effective as emollients in personal care compositions and may be used at a relatively high level in such compositions. The propellant-free foamable transparent compositions according to the invention preferably contain at least 10%, more preferably at least 20%, by weight oil of the water-immiscible oil composition.

Surprisingly, we have found that several siloxane resins, which are insoluble in mineral oil and insoluble in vegetable oil are nevertheless soluble in certain mixtures of mineral oil and vegetable oil. This is also true for many of the preferred MQ resins. In such compositions, the vegetable oil, which is a mixture of triglyceride ester compounds, appears to act as a solubilising agent for the siloxane resin thus making the mineral oil, the siloxane resin and the vegetable oil miscible to form a transparent liquid.

Thus the invention also provides a propellant-free foamable transparent composition, foamable by mixing with air, which comprises a water-immiscible oil composition comprising a miscible blend of at least one mineral oil and at least one vegetable oil and a siloxane resin foam stabiliser, in which compositions the siloxane resin is soluble in neither the mineral oil nor the vegetable oil individually but is soluble in the miscible blend of mineral oil and vegetable oil to form a transparent liquid. The preferred amounts of mineral oil and vegetable oil used to obtain such a transparent liquid blend are from 40 or 50% up to 90 or 95% by weight mineral oil and from 50 or 60% down to 5 or 10% vegetable oil, based on the total weight of water-immiscible oil present in the composition according to the invention.

Propellant-free foamable transparent compositions according to the invention have a wide variety of potential uses, including personal care products such as hair care compositions, for example styling foams, oil moisturisers, cuticle coat, rinse-off and leave-on hair conditioners; depilatory compositions, for example depilatory foam, shaving foam for men and women, pre-shave treatments and aftershaves; deodorant compositions, for example moisturising antiperspirant; sun care compositions, for example sunscreen foaming oil; men's grooming compositions, for example moisturising facial foam, facial cleanser, bath foaming oil or shower oil for men; colour cosmetic compositions, for example foaming foundation; fragrances, for example foaming fragrant oil for aromatherapy; wipes; health care products such as rectal cleaning foam and wound care treatments; or household care products such as wood and hard surface treatments, for example lubricants and cleansers. Such compositions may contain additional ingredients as appropriate, for example colorants, UV absorbers, antiperspirants, fragrances, antibacterial or antifungal agents, pigments, preservatives, sunscreens, vitamins and/or vegetable extracts. Some of the additional ingredients may cause the transparency of the propellant-free foamable composition to be altered either permanently or temporarily. For example, pigments may be included which could provide a transparent composition after sedimentation and may be re-suspended through agitation prior to pumping. Such non-transparent compositions are also included within the scope of this invention provided the water-immiscible oil composition and siloxane resin with optional solubilizer are able to form a transparent liquid in the absence of such additional ingredient.

The invention accordingly also comprises cosmetic compositions comprising in a cosmetic acceptable medium a propellant-free foamable transparent composition according to the first aspect of this invention.

The invention is illustrated by the following examples, in which parts and percentages are by weight.

### EXAMPLES 1 TO 3

70 parts siloxane resin having trimethyl siloxane groups (M) and SiO₂ groups (Q) in an average M/Q number ratio of 0.72/1 were dissolved in 30 parts octyl stearate. The resulting siloxane resin solution was incorporated into three foamable oil-based skin care compositions having the formulations shown in Table 1, in amounts also shown in Table 1. In all formulations the siloxane resin remained dissolved in the water-immiscible oil composition to form a transparent liquid.

TABLE 1

| | Example 1 | Example 2 | Example 3 |
|---|---|---|---|
| Mineral Oil, emollient | 66.5% | 64.15% | 23.3% |
| Crodamol AB C12-15 alkyl | 23.3% | | |
| benzoate, emollient ester | | | |
| Finsolv TN C12-15 alkyl benzoate, | | | 66.5% |
| emollient | | | |
| Isopropyl palmitate, emollient | | 17.82% | |
| Dow Corning 556 Phenyl | | 10.69% | |
| trimethicone silicone fluid, | | | |
| skin feel enhancer | | | |
| Fragrance | 0.2% | 0.21% | 0.2% |
| Siloxane resin solution | 10.0% | 7.13% | 10.0% |

Crodamol, Finsolv and Dow Corning are trademarks

Each of the compositions of Examples 1 to 3 was packed in packaging using a mechanical pump which incorporates air into the liquid material upon activation. When dispensed with this pump action, each composition formed a rich and stable foam.

### EXAMPLES 4 TO 8

The siloxane resin solution prepared in Example 1 was incorporated into various oil-based foamable compositions as shown in Table 2, in amounts also shown in Table 2. Each composition was a transparent liquid and formed a stable foam when dispensed from packaging using a mechanical pump which incorporates air into the liquid material upon activation.

TABLE 2

| | | | | | Example 8 - |
|---|---|---|---|---|---|
| | Example 4 - | | | Example 7 - | white |
| | Moisturising | Example 5 - | Example 6 - | anhydrous | anhydrous |
| | Antiperspirant | Sunscreen | Sunscreen | foaming | foaming |
| | foam | foaming oil | foaming oil | foundation | foundation |
| Tegosoft TN emollient | 60% | 60% | 65% | 69.8% | 69.5% |
| ester | | | | | |
| Mineral oil | 20% | 20% | 20% | 20% | 20% |
| Siloxane resin solution | 15% | 10% | 10% | 10% | 10% |
| Reach 103 | 5% | | | | |
| antiperspirant | | | | | |
| UV A and B sunscreens | | 10% | | | |
| Eusolex 9020 sunscreen | | | 5% | | |
| Red, brown or yellow | | | | 0.2% | |
| pigment | | | | | |
| Unipure LC981 white | | | | | 0.5% |
| pigment | | | | | |

Tegosoft, Reach, Eusolex ® and Unipure are trademarks.

### EXAMPLES 9 TO 11

The siloxane resin solution of Example 1 was added to various compositions comprising vegetable oil and/or mineral oil at a level of 10% resin solution (7.5% siloxane resin based on oil). The resin solubility was noted and is shown in Table 3 below. Examples 9 to 11 are examples according to this invention in which the resin is soluble in the oil composition. In Comparative Examples C1 to C3, the resin is insoluble in the oil composition and immiscible therein in the compositions made.

Each of the compositions of Examples 9 to 11 and Comparative Examples C1 to C3 was dispensed using a mechanical pump which incorporates air into the liquid material. The foaminess of the dispensed composition after standing in air for a minute is noted in Table 3.

TABLE 3

| Example | | | | Foaminess after |
|---|---|---|---|---|
| No. | Oil composition | Resin solubility | Composition aspect | 1 minute |
| C1 | 100% rape seed oil | Highly insoluble | Gelly appearance, | Not foamy |
| | | | opaque | |
| C2 | 75% rape seed oil, 25% | Insoluble | Opaque | Not foamy |
| | mineral oil | | | |
| 9 | 50% rape seed oil, 50% | Soluble | Very slight haze | Foamy |
| | mineral oil | | | |
| 10 | 25% rape seed oil, | Soluble | Water clear | Foamy |
| | 75% mineral oil | | | |
| 11 | 10% rape seed oil, | Soluble | Water clear | Foamy |
| | 90% mineral oil | | | |
| C3 | 100% mineral oil | Insoluble | Cloudy, almost | Foamy |
| | | | opaque | |

Table 3 shows that although the siloxane resin is insoluble in aliphatic oil and in rape seed oil, it may be made soluble in a blend of rape seed oil and mineral oil in the range of weight ratio mineral oil/rape seed oil 50/50 to mineral oil/rape seed oil 90/10.

### EXAMPLE 12

An oil-based Formulation A comprising 70 parts Tegosoft TN and 20 parts aliphatic oil was prepared containing no foaming agent. 10 parts of the siloxane resin solution of Example 1 was added to Formulation A to produce the composition of Example 12. As a comparison, 10 parts ‘SPAN 80’ (trademark) sorbitan oleate, was added to Formulation A to produce Formulation B. Sorbitan esters have been proposed in U.S. Pat. No. 4,639,367 as foaming agents for an anhydrous aerosol foam.

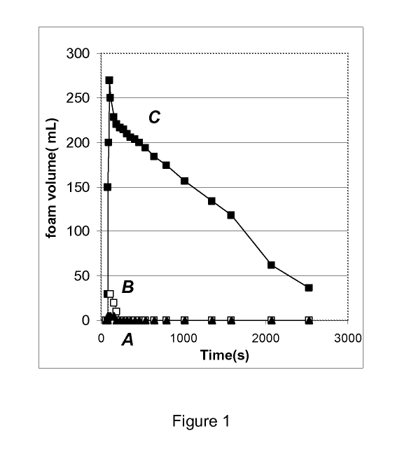

Formulations A and B and the composition of Example 12 were tested by a protocol comprising incorporating air in the oil compositions and recording the foam volume as a function of time. 50 ml of the oil composition are placed in a cylinder of which volume is about 350 ml. Air is incorporated in the formulation thanks to a gas diffuser (chalk-made gas diffuser) which is placed in the liquid (a flow rate of 1.1 l/min is used). Once the air starts to blow into the oil formulation, some foam is generated. The air diffuser is removed after 40 seconds. If the foam is stable enough, the volume of foam reaches a value of 300 ml. Foam level is recorded as a function of time. FIG. 1 is a graph comparing the foaming tendency of the three formulations (the composition of Example 12 is marked C).

Formulation A produces very unstable foam. Foam volume is limited during the 40 seconds of blowing, and the foam collapses as soon as air flow is stopped. Once air is incorporated into formulation B, more foam is created, which is also more stable. Once air is incorporated in Formulation C (Example 12), volume of foam increases constantly with air incorporation. Once the air flow is stopped, the foam is also much more stable, as seen in the graph of FIG. 1.

### EXAMPLE 13 AND 14

Example 13 was prepared using 10.0 parts of the siloxane resin solution prepared in Example 1 incorporated into an oil-based foamable composition comprising 79.8 parts ‘Gemseal 25’ volatile C₁₃₋₁₅ alkane, 0.2 parts fragrance and 10.0 parts vegetable oil blend to give a transparent liquid composition which formed a stable foam when dispensed from packaging using a mechanical pump which incorporates air into the liquid material. Example 14 was prepared using 20.0 parts of the siloxane resin solution prepared in Example 1 incorporated into an oil-based foamable composition comprising 80.0 parts ‘Gemseal 25’ volatile C13-15 alkane to give a transparent liquid composition which formed a stable foam when dispensed from packaging using a mechanical pump which incorporates air into the liquid material.

### EXAMPLE 15

10.0 parts of the siloxane resin solution prepared in Example 1 were incorporated into an oil-based foamable composition comprising 50.0 parts C₁₂₋₁₅ alkyl benzoate and 20.0 parts mineral oil, 10.0 parts caprylic/capric triglyceride and sunscreen actives: 6.0 parts ethylhexyl methoxycinnamate, 2.0 parts 4-methylbenzylidene camphor, 2.0 parts benzophenone-3 to give a transparent liquid composition which formed a stable foam when dispensed from packaging using a mechanical pump which incorporates air into the liquid material.

### EXAMPLE 16

A coloured composition was prepared by incorporating 10.0 parts of the siloxane resin solution prepared in Example 1, into 70.0 parts of emollient ester (C12-15 Alkyl benzoate) and 19.9 parts mineral oil and 0.1 parts of oil soluble dyes. The compositions were transparent coloured liquid compositions which formed a stable coloured foam when dispensed from packaging using a mechanical pump which incorporates air into the liquid material.

### COMPARATIVE EXAMPLE C4

20 parts of a siloxane resin B having D units (dimethyl siloxane units and diphenyl siloxane units) and T units (methyl siloxane units and phenyl siloxane units) in an average D/T number ratio of 0.25/1 were incorporated into 80 parts of C₁₃₋₁₅ alkane volatile solubilising agent without additional ingredient to form a non-homogeneous oil composition. The siloxane resin was not miscible with the chosen solubilising agent.

### EXAMPLES 17 TO 19 AND COMPARATIVE EXAMPLE C5

A siloxane resin C having trimethyl siloxane groups (M) and SiO₂ groups (Q) in an average M/Q number ratio of 0.80/1, was incorporated into foamable oil-based skin care compositions comprising Gemseal 25 according to the formulations shown in Table 4. In Comparative Example C5, the siloxane resin C was only partially miscible with the solubilizer and did therefore not provide a transparent liquid, but a stable foam was formed when dispensed from packaging using a mechanical pump which incorporates air into the liquid material. In Examples 17 to 19, the addition of a vegetable oil blend of rape seed oil and palm oil allows for the water-immiscible oil compositions to form transparent liquid compositions which formed stable foam when dispensed from packaging using a mechanical pump which incorporates air into the liquid material.

TABLE 4

| | | | Exam- | Exam- |
|---|---|---|---|---|
| | | | ple 18 | ple 19 |
| | Comparative | Example 17 | Styling | Styling |
| | Example C5 | Styling foam | foam | foam |
| Gemseal 25, C13-15 | 80% | 72% | 67% | 67% |
| alkane | | | | |
| Vegetable oil blend | | 18% | 20% | 20% |
| of rape seed oil and | | | | |
| palm oil | | | | |
| HY 4008 vegetable | | | | |
| oil blend | | | | |
| Brassica Campestris | | | | |
| (Rapeseed) Seed | | | | |
| Oil and Elaeis | | | | |
| Guineensis (Palm) | | | | |
| Oil | | | | |
| Siloxane resin C | 20% | 10% | 10% | 10% |
| Dow Corning 2502 | | | 3% | |
| cosmetic fluid, Cetyl | | | | |
| Dimethicone | | | | |
| Dow Corning 2503 | | | | 3% |
| cosmetic wax, | | | | |
| Stearyl Dimethicone | | | | |

Gemseal and Dow Corning are trademarks.

### EXAMPLES 20 TO 25

The siloxane resin solution of Example 1 was incorporated into three foamable oil-based skin care compositions having the formulations shown in Table 5, in amounts also shown in Table 5. In all formulations the siloxane resin remained dissolved in the water-immiscible oil composition to form a transparent liquid.

TABLE 5

| | Example 20 | Example 21 | Example 22 | Example 23 | Example 24 | Example 25 |
|---|---|---|---|---|---|---|
| | Face | Face | After-shave | Bath | Aromatherapy | Aromatherapy |
| | care oil | care oil | foaming oil | foaming oil | foaming oil | foaming oil |
| Tegosoft TN | 68% | 68% | 68% | 60% | 66.5% | 67% |
| C12-15 Alkyl Benzoate | | | | | | |
| Mineral oil | 20% | 20% | 20% | 20% | 20.0% | 20% |
| Siloxane resin solution | 10% | 10% | 10% | 10% | 10.0% | 10% |
| Vegetol Aloes, Mineral | 2% | | | | | |
| Oil and Vegetable Oil | | | | | | |
| and Aloe Ferox Leaf | | | | | | |
| Extract | | | | | | |
| Gatuline Derma- | | 2% | | | | |
| sensitive, Octyldodecyl | | | | | | |
| Myristate (and) | | | | | | |
| Capparis Spinosa Fruit | | | | | | |
| Extract | | | | | | |
| Depiline, | | | 2% | | | |
| Caprylic/Capric | | | | | | |
| Triglyceride (and) | | | | | | |
| Tribehenin (and) | | | | | | |
| Sorbitan Laurate (and) | | | | | | |
| Nylon-12 (and) | | | | | | |
| Palmatine | | | | | | |
| Myritol 318, | | | | 8% | | |
| Caprylic/Capric | | | | | | |
| Triglyceride | | | | | | |
| V-Tonic, Mentha | | | | 2% | | 3% |
| Arvensis Leaf Oil and | | | | | | |
| Citrus Medica Limonum | | | | | | |
| (Lemon) Peel Oil and | | | | | | |
| Cupressus | | | | | | |
| Sempervirens | | | | | | |
| Leaf/Nut/Stem Oil and | | | | | | |
| Lavandula Hybrida Oil | | | | | | |
| and Cistus Ladaniferus | | | | | | |
| Oil | | | | | | |
| Essential oil Ylang- | | | | | 3.5% | |
| Ylang | | | | | | |

Tegosoft, Vegetol, Gatuline, Depiline, V-Tonic are trademarks.

Each of the compositions of Examples 20 to 25 was packed in packaging using a mechanical pump which incorporates air into the liquid material upon activation. When dispensed with this pump action, each composition formed a stable foam.

1. A propellant-free foamable transparent composition, foamable by mixing with air, comprising a water-immiscible oil composition comprising at least one water-immiscible oil and a siloxane resin foam stabilizer, which is miscible with the water-immiscible oil composition to form a transparent liquid.

2. A propellant-free foamable transparent composition according to claim 1, wherein the water immiscible oil is selected from one or more mineral oils, one or more vegetable oils and a mixture of one or more mineral oils and one or more vegetable oils.

3. A propellant-free foamable transparent composition according to claim 1, wherein the siloxane resin foam stabiliser is soluble in the water-immiscible oil composition to form a transparent liquid.

4. A propellant-free foamable transparent composition according to claim 1, which also comprises a solubilising agent for the siloxane resin foam stabiliser, wherein the water-immiscible oil composition, the siloxane resin foam stabiliser and the solubilising agent are miscible to form a transparent liquid.

5. A propellant-free foamable transparent composition according to claim 4, wherein the siloxane resin and solubilising agent are present in the composition in a weight ratio in the range 1:25 to 5:1.

6. A propellant-free foamable transparent composition according to claim 4, wherein the solubilising agent is selected from one or more of an alkyl ester of an aliphatic carboxylic acid having 12 to 20 carbon atoms, octyl stearate, an alkyl ester of a carboxylic acid in which the alkyl group has 12 to 20 carbon atoms, an alcohol having 12 to 20 carbon atoms.

7. A propellant-free foamable transparent composition according to claim 4, wherein the silicone resin foam stabiliser and the solubilising agent are present in the composition in a weight ratio in the range 1:25 to 5:1

8. A propellant-free foamable transparent composition according to claim 1, wherein the water-immiscible oil composition comprises a miscible blend of one or more mineral oils and one or more vegetable oils, the siloxane resin foam stabiliser being insoluble in any of the mineral oils and in any of the vegetable oils individually but being soluble in the miscible blend of mineral oil and vegetable oil to form a transparent liquid

9. A propellant-free foamable transparent composition according to claim 8, wherein the mineral oil(s) and vegetable oil(s) are present in the composition in a weight ratio in the range of from 50:50 to 90:10.

10. A propellant-free foamable transparent composition according to claim 1 wherein the siloxane resin foam stabiliser consists predominantly of monovalent trihydrocarbonsiloxy (M) groups of the formula R″₃SiO_{1/2} and tetravalent (Q) groups of the formula SiO_{4/2} wherein R″ denotes a monovalent hydrocarbon group and the number ratio of M groups to Q groups is in the range 0.4:1 to 1.2:1.

11. A propellant-free foamable transparent composition according to claim 1, wherein the siloxane resin foam stabiliser is present in an amount of from 2 to 20% by weight of the propellant-free foamable transparent composition.

12. A propellant-free foamable transparent composition according to claim 2, wherein any mineral oil is an aliphatic oil or a hydrocarbon polymer and any vegetable oil is selected from coconut oil, groundnut oil, soybean oil, sunflower oil, olive oil, hydrogenated castor oil, sweet almond oil, calophyllum, palm oil, castor oil, avocado oil, jojoba oil, cereal germ oil, corn oil, cottonseed oil, walnut oil, linseed oil, mink oil, illipe butter, rapeseed oil, soybean oil, sunflower seed oil, blackcurrant oil, borage oil, camelina oil, evening primrose oil, rice bran oil and flaxseed oil

13. A propellant-free foamable transparent composition according to claim 1 wherein the silicone resin is present at 2 to 20% by weight of the foamable composition.

14. A cosmetic composition comprising in a cosmetic acceptable medium a propellant-free foamable transparent composition as claimed in claim 1.

15. (canceled)
